(19) United States
(12) Patent Application Publication
(10) Pub. No.: US 2015/0258078 A1
(43) Pub. Date: Sep. 17, 2015

(54) COMPOSITIONS CONTAINING BERBERINE AND PANTETHINE AND METHODS FOR TREATMENT OF LIPID METABOLISM DISORDERS

(71) Applicant: Alexander R. Shikhman, Escondido, CA (US)

(72) Inventor: Alexander R. Shikhman, Escondido, CA (US)

(21) Appl. No.: 14/206,208

(22) Filed: Mar. 12, 2014

(51) Int. Cl. A61K 31/4375; A61K 31/4525; A61K 31/4188; A61K 31/12; A61K 31/16; A61K 33/24

(52) U.S. Cl. CPC A61K 31/4375; A61K 31/16; A61K 33/24; A61K 31/4188; A61K 31/12; A61K 31/4525

(57) ABSTRACT

The present invention is directed to compositions incorporating purified herbal ingredients for the treatment of conditions such as hypercholesterolemia, hyperlipidemia, elevated triglycerides, syndrome X (metabolic syndrome), hyperglycemia, diabetes, cardiovascular disease and the like as well as disorders of lipid metabolism with failed response to standard therapies (for example statins) or any other disease or condition associated with lipids and lipid metabolism, as well as methods for their use. The compositions incorporating purified herbal ingredients can incorporate combinations of berberine and pantethine, alone or with one or more of chromium polynicotinate or chromium picolinate, biotin, curcumin and/or bioperin.

### PRIORITY CLAIM

This application claims the benefit of U.S. Provisional Application Ser. No. 61/784,055 filed Mar. 14, 2013, the substance of which is incorporated herein in its entirety.

### FIELD OF THE INVENTION

This invention is directed to compositions and methods for the treatment lipid metabolism disorders, such as hypercholesterolemia, hyperlipidemia, elevated triglycerides, syndrome X (metabolic syndrome), hyperglycemia, diabetes, cardiovascular disease and the like as well as disorders of lipid metabolism with failed response to standard therapy (for example statins). In particular the present invention involves compositions that include berberine and pantethine, and optionally include chromium polynicotinate or chromium picolinate, biotin, curcumin and/or bioperin.

### BACKGROUND

Cardiovascular disease, including coronary artery disease, atherosclerosis, cerebral vascular disease, cerebral vascular accident (stroke), myocardial infarction, sudden death syndrome, is the number one cause of death in most developed countries all over the world. Elevated circulating cholesterol levels, in particular low-density-lipoprotein cholesterol (LDL-cholesterol) levels, have been well established as one of the major risk factors for the development and progression of cardiovascular and cerebral vascular diseases, as have high levels of circulating lipids such as triglycerides. In addition, these subjects often also experience obesity, metabolic disorders (such as syndrome X and diabetes) and hyperlipidemia, which are all major subgroups of the population that are adversely affected by high cholesterol and triglyceride levels.

Although advances have been made in treating cardiovascular disease and metabolic disorders associated with hyperlipidemia, hypercholesterolemia and the like, these conditions still are responsible for significant deterioration of the quality of life and risk of death for many patients. In many cases, medications used to treat these conditions are not well tolerated and have significant side effects. For example, the major drawback to the predominant statin or statin-like compounds is muscle soreness, muscle weakness, muscle tenderness, intense muscle pain (collectively known as statin-induced myopathy), peripheral neuropathy and extreme form of muscle damage called rhabdomylosis. Rhabdomylosis can be both a serious and a life threatening side effect clearly associated with the use of statin drugs where the muscle breakdown causes major organ damage to both the liver and kidney that has resulted in many reported deaths.

Berberine is a quaternary ammonium salt from the protoberberine group of isoquinoline alkaloids. It is found in such plants as Berberis [e.g. Berberis aquifolium (Oregon grape), Berberis vulgaris (barberry), and Berberis aristata (tree turmeric)], Hydrastis canadensis (goldenseal), Phellodendron amurense (Amur cork tree, huang bai, huang po, po mu) and Coptis chinensis (Chinese goldthread, huang-lian, huang-lien), and Tinospora cordifolia, and to a smaller extent in Argemone mexicana (prickly poppy) and Eschscholzia californica (Californian poppy). Berberine is usually found in the roots, rhizomes, stems, and bark.

Berberine is also a nucleic acid-binding isoquinolone alkaloid with wide potential therapeutic properties. In this regard, berberine is known as a traditional medicine or dietary supplement, with some activity against fungal infections (for example, infection due to Candida albicans), parasites, and bacterial/viral infections. Berberine is also considered to have anti-inflammatory properties and modifies production of certain proinflammatory cytokines and E-selectin. It also increases adiponectin expression which partly explains its versatile health effects. Further, various studies have studies have shown berberine has various beneficial effects on the cardiovascular system, including lipid-lowering activities. See, e.g., International Patent Application Number WO2007113748A1; International Patent Application Number WO2007090289A1; European Patent Application Number EP0813871A1; Expert Opin Biol Ther. 2012 August; 12(8):1113-24; Lipids Health Dis. 2012 Jul. 4; 11:86; Phytomedicine. 2012 Jul. 15; 19(10):861-7; Anal Bioanal Chem. 2012 May; 403(3):847-56; Adv Ther. 2011 December; 28(12):1105-13; Eur J Pharmacol. 2011 Jun. 25; 660(2-3):368-74; Nutr Metab Cardiovasc Dis. 2010 November; 20(9):656-61; Metabolism. 2008 August; 57(8):1029-37; Biol Pharm Bull. 2008 June; 31(6):1169-76; Arterioscler Thromb Vasc Biol. 2005 October; 25(10):2170-6; Expert Opin Investig Drugs. 2005 May; 14(5):683-5; Nat Med. 2004 December; 10(12):1344-51; and Expert Opin Ther Pat. 2012 Dec. 12. [Epub ahead of print].

Pantethine (bis-pantethine or co-enzyme pantethine) is a dimeric form of pantothenic acid (vitamin B5). It is composed of two molecules of pantothenic acid linked by cysteamine bridging groups. The monomer of this compound is known as pantetheine and is an intermediate in the production of Coenzyme A by the body. Pantethine is considered the more biologically active form of vitamin B5, but it is less stable, decomposing over time if it is not kept refrigerated. Most vitamin B5 supplements are therefore in the form of calcium pantothenate, a salt of pantothenic acid.

Pantethine is available as a dietary supplement because of evidence of its health benefits, including potential biological activity in the prevention and treatment of pantothenic acid deficiency; wasting disease; hyperthyroidism; hyperlipidemia; atonic constipation; adverse drug reactions caused by streptomycin and kanamycin; acute or chronic eczema; platelet count and bleeding tendency and hypercholesterolemia and cardiovascular disease. See, e.g., European Patent Application Number EP2476408A1; European Patent Application Number EP1547584B1; Minerva Med. 1990 June; 81(6):475-9; Atherosclerosis. 1987 November; 68(1-2):41-9; Angiology. 1987 March; 38(3):241-7; Int J Clin Pharmacol Ther Toxicol. 1986 November; 24(11):630-7; Clin Ther. 1986; 8(5):537-45; Int J Clin Pharmacol Res. 1985; 5(5):309-18; Atherosclerosis. 1984 December; 53(3):255-64; Atherosclerosis. 1984 January; 50(1):73-83; Atherosclerosis. 1982 February; 41(2-3):267-77; and Tokai J Exp Clin Med. 1981 January; 6(1):49-57.

Biotin is a vitamin with a variety of potential therapeutic effects, including in regulation of triglycerides and hypoglycemia. See, e.g., European Patent Application Number EP1462444A1; Biomed Pharmacother. 2006 May; 60(4):182-5; J Nutr Biochem. 2005 July; 16(7):424-7; Nutr Metab. 1976; 20(1):41-61; and J Cardiometab Syndr. 2007 Spring; 2(2):91-7. Curcumin (1) is a natural product isolated in abundance from Curcuma species, in particular, Curcuma longa (Zingiberaceae) Linn. See Park and Kim, /. Nat. Prod. 65: 1227 (2002) and U.S. Pat. No. 5,861,415, and has potential activity as an anti-inflammatory, antiviral, antifungal, antibacterial, anticancer, chemopreventive, in the prevention and treatment of Alzheimer's Disease and dislipidemias. See, e.g., International Patent Application Number WO2007143635A1; U.S. Pat. No. 8,329,757; Mol Nutr Food Res. 2012 May; 56(5):691-701; Phytother Res. 2012 May 21; Mol Nutr Food Res. 2011 December; 55(12):1829-40; Phytother Res. 2011 November; 25(11):1721-6; Nutr Res Pract. 2010 June; 4(3):191-5; Metabolism. 2008 November; 57(11):1576-83; J Nutr Biochem. 2007 February; 18(2):113-9. Epub 2006 May 18; and Acta Med Indones. 2008 October; 40(4):201-10. Likewise, chromium polynicotinate or chromium picolinate may be useful for supplementing dietary chromium, lowering blood glucose levels, lowering serum lipid levels and increasing lean body mass. See, e.g., U.S. Pat. No. 6,432,942; International Patent Application Number WO2000012094A1; J Med. 2000; 31(5-6):227-46; Mol Biol Rep. 2009 July; 36(6):1275-81; J Fam Pract. 1988 December; 27(6):603-6; and J Am Coll Nutr. 1982; 1(3):263-74. Bioperin is another naturally occurring compound found in plants of the Piperaceae family.

Unfortunately, the current therapies for cardiovascular disease, metabolic disorders and the related hypercholesterolemia, hyperlipidemia, elevated triglycerides, syndrome X (metabolic syndrome), hyperglycemia, diabetes and the like are either not effective or have delirious side effects. These treatments are not desirable or suitable for many patients therefore, there is a need for improved compositions and methods to treat these conditions.

### SUMMARY OF THE INVENTION

This invention is based, at least in part, on the discovery that berberine and pantethine have therapeutic properties and when combined exhibit superior and preferentially synergistic effects on the diseases of interest (e.g, diseases of lipid metabolism). These therapeutic properties are useful for the prevention and/or treatment of diseases associated with abnormal lipid metabolism. Thus, the present invention relates to treating conditions in mammals such as hypercholesterolemia, hyperlipidemia, elevated triglycerides, syndrome X (metabolic syndrome), hyperglycemia, diabetes, cardiovascular disease and the like as well as disorders of lipid metabolism with failed response to standard therapies (for example statins) by administering a pharmaceutical composition containing combinations of berberine and pantethine, and optionally one or more of chromium polynicotinate or chromium picolinate, biotin, curcumin and/or bioperin.

Therefore, one embodiment of the present invention is a pharmaceutical composition comprising:

(1) a therapeutically effective quantity of berberine; and

(2) a therapeutically effective quantity of pantethine, in a unit dose.

Typically, for this embodiment and other embodiments that are pharmaceutical compositions, the pharmaceutical composition further comprises a pharmaceutically acceptable carrier, diluent, or excipient.

In general, the pharmaceutical composition typically comprises from about 50 mg to about 1000 mg of berberine and from about 50 mg to about 2000 mg of pantethine as a unit dose.

The pharmaceutical composition can be formulated for oral, transcutaneous, parenteral, or intraarticular routes of administration. Typically, the pharmaceutical composition is formulated for the treatment of a disease or condition selected from the group consisting of hypercholesterolemia, hyperlipidemia, elevated triglycerides, syndrome X (metabolic syndrome), hyperglycemia, diabetes, cardiovascular disease and the like as well as disorders of lipid metabolism with failed response to standard therapies (for example statins).

Another embodiment of the present invention, as described above, includes one or more additional compounds selected from the group consisting of chromium polynicotinate or chromium picolinate, biotin, curcumin and/or bioperin in a therapeutically effective quantity in the unit dose. In general, this embodiment comprises:

(1) a therapeutically effective quantity of berberine;

(2) a therapeutically effective quantity of pantethine; and

(3) a therapeutically effective quantity of one or more of chromium polynicotinate, chromium picolinate, biotin, curcumin or bioperin in a unit dose.

Typically, the therapeutically effective quantity of chromium polynicotinate or chromium picolinate is from about 1 mcg to about 500 mcg, the therapeutically effective quantity of biotin is from about 0.1 mg to about 50 mg, the therapeutically effective quantity of curcumin is from about 50 mg to about 2000 mg, and the therapeutically effective amount of bioperin is typically from about 0.1 mg to about 20 mg per unit dose.

Still another embodiment of the present invention is a purified preparation of at least 5% berberine and at least 5% pantethine.

Still another embodiment of the present invention is a method for treating a patient who has a condition selected from the group consisting of hypercholesterolemia, hyperlipidemia, elevated triglycerides, syndrome X (metabolic syndrome), hyperglycemia, diabetes, cardiovascular disease and the like as well as disorders of lipid metabolism with failed response to standard therapies (for example statins) comprising the step of administering a therapeutically effective dose of a pharmaceutical composition according to the present invention, as described above, to the patient to treat the patient.

Unless otherwise defined, all technical and scientific terms used herein have the same meaning as commonly understood by one of ordinary skill in the art to which this invention belongs. Methods and materials are described herein for use of the present invention; other suitable methods and materials known in the art can also be used. The materials and methods, and examples are illustrative only and not intended to be limiting. All publications, patent applications, patents and other references mentioned herein, are incorporated by reference in their entirety. In case of conflict, the present specification, including definitions will control.

These, and other, embodiments of the invention will be better appreciated and understood when considered in conjunction with the following description and the accompanying drawings. It should be understood, however, that the following description, while indicating various embodiments of the invention and numerous specific details thereof is given by way of illustration and not of limitation. Many substitutions, modifications, additions and/or rearrangements may be made within the scope of the invention without departing from the spirit thereof and the invention includes all such substitutions, modifications, or additions.

### DETAILED DESCRIPTION OF THE INVENTION

In accordance with the present invention and as used herein, the following terms are defined with the following meanings, unless explicitly stated otherwise. These explanations are intended to be exemplary only. They are not intended to limit the terms as they are described or referred to throughout the specification. Rather, these explanations are meant to include any additional aspects and/or examples of the terms as described and claimed herein.

The following terms are used herein:

The term “active ingredient” refers to a therapeutically effective amount of drug, natural remedy or foundation thereof preferably, active ingredients of the present invention are berberine, pantethine and, optionally including, chromium polynicotinate, chromium picolinate, biotin, curcumin or bioperin.

The term “therapeutically effective amount” refers to the amount of an active ingredient necessary to induce one or more of the desired pharmacological effects of the current invention. The amount can vary greatly according to the effectiveness of a particular active substance; the age, weight, and response of the individual; as well as the nature and severity of the individual's symptoms. Accordingly, there is no upper or lower critical limitation with respect to the amount of the active substance. A therapeutically effective amount to be employed in the present invention can readily be determined by those skilled in the art.

The term “in combination with” as used herein means that the described agents can be administered to a subject together in a mixture, concurrently or as a single agents or sequentially as single agents in any order.

The term “preventing” refers to reducing the likelihood that the recipient will incur or develop any of the pathological conditions described herein.

The term “subject” means any mammal including humans.

The term “pharmaceutical” refers to a compound of composition that, when administered to a subject, provides one or more therapeutic or prophylactic benefits to said subject. As used herein, “pharmaceutical” includes nutraceuticals, natural products, natural remedies, synthetic chemicals and any other biologically active compounds or compositions according to the present invention.

The term “treating” refers to mediating a disease or condition and preventing, or mitigating, its further progression or ameliorate the symptoms associated with the disease or condition.

Methods and materials are described herein. However, methods and materials similar or equivalent to those described herein can be also used to obtain variations of the present invention. The materials, methods, and examples are illustrative only and not intended to be limiting.

It has been discovered that the herbal ingredients berberine and pantethine have unique therapeutic properties and more particularly have synergistic therapeutic effect. The present invention is directed to compositions incorporating these purified herbal ingredients for the treatment of conditions such as hypercholesterolemia, hyperlipidemia, elevated triglycerides, syndrome X (metabolic syndrome), hyperglycemia, diabetes, cardiovascular disease and the like as well as disorders of lipid metabolism with failed response to standard therapies (for example statins), as well as methods for their use. The compositions incorporating purified herbal ingredients can incorporate the following optional ingredients in addition to berberine and pantethine: chromium polynicotinate, chromium picolinate, biotin, curcumin or bioperin.

Berberine is a biologically active substance present in such plants as in such plants as Berberis [e.g. Berberis aquifolium (Oregon grape), Berberis vulgaris (barberry), and Berberis aristata (tree turmeric), Hydrastis canadensis (goldenseal), Phellodendron amurense (Amur cork tree, huang bai, huang po, po mu) and Coptis chinensis (Chinese goldthread, huang-lian, huang-lien), and Tinospora cordifolia, and to a smaller extent in Argemone mexicana (prickly poppy) and Eschscholzia californica (Californian poppy). Berberine is usually found in the roots, rhizomes, stems, and bark.

The structural formula of berberine is Formula (I), below:

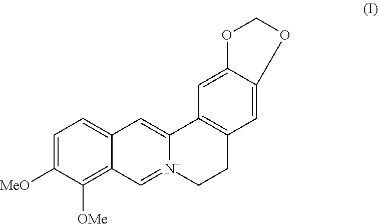 [SMILES: COc1ccc2cc3[n+](cc2c1OC)CCc1cc2c(cc1-3)OCO2]

As a traditional medicine or dietary supplement, berberine has shown some activity against fungal infections (for example, infection due to Candida albicans), parasites, and bacterial/viral infections. Berberine is considered antibiotic. When applied in vitro and in combination with methoxyhydnocarpin, an inhibitor of multidrug resistance pumps, berberine inhibits growth of Staphylococcus aureus and Microcystis aeruginosa, a toxic cyanobacterium. Berberine is also considered to have anti-inflammatory properties and modifies production of certain proinflammatory cytokines and E-selectin. It also increases adiponectin expression which partly explains its versatile health effects. Berberine is a nucleic acid-binding isoquinolone alkaloid with wide potential therapeutic properties.

During the last few decades, many studies have shown berberine has various beneficial effects on the cardiovascular system and anti-inflammatory activities. A Canadian report suggested berberine can effectively reduce intracellular superoxide levels in LPS-stimulated macrophages. Such a restoration of cellular redox by berberine is mediated by its selective inhibition of gp91 phox expression and enhancement of SOD activity.

Berberine exerts up-regulating activity on both the low-density-lipoprotein receptor (LDLR) and the insulin receptor (InsR). In addition, berberine has been tested and used in experimental treatment of diabetes mellitus. Further, berberine has been shown to lower elevated blood glucose as effectively as metformin. The mechanisms of action include inhibition of aldose reductase, inducing glycolysis, preventing insulin resistance through increasing insulin receptor expression and acting like incretins. A new study suggested berberine may overcome insulin resistance via modulating key molecules in insulin signaling pathway, leading to increased glucose uptake in insulin-resistant cells.

Berberine might exert its insulinotropic effect in isolated rat islets by up-regulating the expression of hepatocyte nuclear factor 4 alpha, which probably acts solely or together with other HNFs to modulate glucokinase activity, rendering β cells more sensitive to glucose fluctuation and to respond more effectively to glucose challenge.

Berberine seems to inhibit human dipeptidyl peptidase-4 (DPP IV), as well as the prodiabetic target human protein tyrosine phosphatase 1B, which explain at least some of its antihyperglycemic activities. Berberine suppresses intestinal disaccharidases with beneficial metabolic effects in diabetic states. A recent comprehensive metabonomics method, applied to 60 type 2 diabetics, suggested administration of berberine down-regulates the high level of free fatty acids which are known to be toxic to the pancreas and cause insulin resistance. These results suggest berberine might play a role in the treatment of type 2 diabetes Further, berberine has been shown to boost the effects of metformin and 2,4-thiazolidinedione (THZ), and can partly replace the commercial drugs, which could lead to a reduction in toxicity and side effects of the latter.

Berberine inhibits Foxo1, which integrates insulin signaling with mitochondrial function. Inhibition of Foxo1 can improve hepatic metabolism during insulin resistance and the metabolic syndrome.

Berberine lowers elevated blood total cholesterol, LDL cholesterol, triglycerides and atherogenic apolipoproteins (apo B) (Apo B), but the mechanism of action is distinct from statins. Berberine reduces LDL cholesterol by upregulating LDLR mRNA expression posttranscriptionally while downregulating the transcription of proprotein convertase subtilisin/kexin type 9 (PCSK9), a natural inhibitor of LDL receptor (LDLR), and increasing in the liver the expression of LDL receptors through extracellular signal-regulated kinase (ERK) signaling pathway, while statins inhibit cholesterol synthesis in the liver by blocking HMG-CoA-reductase. This explains why berberine does not cause side effects typical to statins. Berberine and plant stanols synergistically inhibit cholesterol absorption in hamsters.

Berberine seems to improve the arterial endothelial function in humans. Berberine activates AMP-activated protein kinase (AMPK), specifically extracellular signal-regulated kinases (ERK), which plays a central role in glucose and lipid metabolism, suppresses proinflammatory cytokines, and reduces MMP-9 and EMMPRIN expression, which are all beneficial changes for heart health. Moreover, berberine reduces hepatic fat content in the rats of nonalcoholic fatty liver disease. Berberine also prevents proliferation of hepatic stellate cells (HSCs), which are central for the development of fibrosis during liver injury.

Pantethine (bis-pantethine or co-enzyme pantethine) is a dimeric form of pantothenic acid (vitamin B5). It is composed of two molecules of pantothenic acid linked by cysteamine bridging groups. The monomer of this compound is known as pantetheine and is an intermediate in the production of Coenzyme A by the body. Pantethine is considered the more biologically active form of vitamin B5, but it is less stable, decomposing over time if it is not kept refrigerated. Most vitamin B5 supplements are therefore in the form of calcium pantothenate, a salt of pantothenic acid.

The structural formula of pantethine phosphate ester is Formula (I), below:

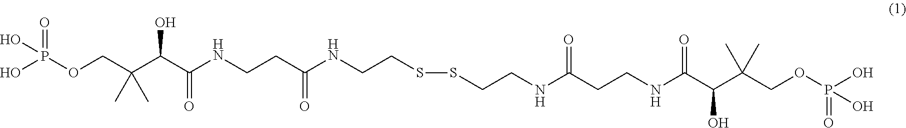 [SMILES: CC(C)(COP(=O)(O)O)[C@@H](O)C(=O)NCCC(=O)NCCSSCCNC(=O)CCNC(=O)[C@H](O)C(C)(C)COP(=O)(O)O]

Pantethine may be useful for: (a) prevention and treatment of a pantothenic acid deficiency; (b) replenishment of pantothenic acid to patients suffering from wasting diseases or hyperthyroidism, or to pregnant and parturient women or breast-feeding women who have an increased demand for pantothenic acid that cannot be supplied sufficiently from foods; and (c) prevention and treatment of hyperlipidemia, atonic constipation, and side effects of streptomycin and kanamycin, improvement of acute and chronic eczema, and improvement in platelet counts and hemorrhagic tendency in blood dyscrasia, when these diseases, disorders or symptoms are presumed to be attributable to a deficiency or a metabolic disorder of pantothenic acid.

Pantethine is available as a dietary supplement because of evidence of its health benefits. In multiple clinical trials of patients with elevated cholesterol and triglycerides, total and LDL cholesterol were decreased by 12%, triglycerides decreased by 18%, and HDL cholesterol was increased by 9%. These clinical trials were conducted with daily intakes ranging from 600 to 1200 mg/day. Within this dose range there is no evidence of a dose-effect relationship, i.e. changes in lipid concentrations overlapped across the range of doses. Direct dose-response evidence is not available because no trial tested more than one dose. A few trials tested 300 mg/day with more modest but still statistically significant results. Further carefully controlled trials of 600 and 900 mg/d doses have shown statistically significant lowering of LDL cholesterol in individuals with greatly or moderately elevated levels of blood lipids.

Although pantethine can serve as a precursor for generation of vitamin B5, this is not thought to be the mechanism of action. Vitamin B5 requirements are on the order of 5 mg/day. Two mechanisms of action are proposed for pantethine. In the first, pantethine serves as the precursor for synthesis of coenzyme A. In the second, pantethine is converted to two pantetheine molecules which are in turn metabolized to form two pantethenic acid and two cysteamine molecules. Cysteamine is theorized to bind to and thus inactivate sulfur-containing amino acids in liver enzymes involved in the production of cholesterol and triglycerides.

Biotin is a vitamin useful as an additive for feed, medicine, etc., and as a process for preparing the same, it has been known a process, for example, in which a thienoimidazol compound represented by the following formula:

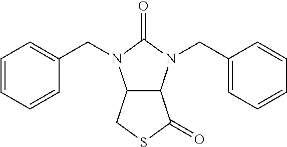 [SMILES: O=C1SCC2C1N(Cc1ccccc1)C(=O)N2Cc1ccccc1]

Biotin is a water-soluble vitamin that acts as a prosthetic group of carboxylases. Besides its role as carboxylase prosthetic group, biotin regulates gene expression and has a wide repertoire of effects on systemic processes. The vitamin regulates genes that are critical in the regulation of intermediary metabolism: Biotin has stimulatory effects on genes whose action favors hypoglycemia (insulin, insulin receptor, pancreatic and hepatic glucokinase); on the contrary, biotin decreases the expression of hepatic phosphoenolpyruvate carboxykinase, a key gluconeogenic enzyme that stimulates glucose production by the liver. The findings that biotin regulates the expression of genes that are critical in the regulation of intermediary metabolism are in agreement with several observations that indicate that biotin supply is involved in glucose and lipid homeostasis. Biotin deficiency has been linked to impaired glucose tolerance and decreased utilization of glucose. On the other hand, the diabetic state appears to be ameliorated by pharmacological doses of biotin. Likewise, pharmacological doses of biotin appear to decrease plasma lipid concentrations and modify lipid metabolism. The effects of biotin on carbohydrate metabolism and the lack of toxic effects of the vitamin at pharmacological doses suggest that biotin could be used in the development of new therapeutics in the treatment of hyperglycemia and hyperlipidemia, an area that we are actively investigating.

Several studies have reported a relationship between biotin and blood lipids. The effect of biotin administration on the concentration of plasma lipids, as well as glucose and insulin in type 2 diabetic and nondiabetic subjects was investigated. Eighteen diabetic and 15 nondiabetic subjects aged 30-65 were randomized into two groups and received either 61.4 micromol/day of biotin or placebo for 28 days. Plasma samples obtained at baseline and after treatment were analyzed for total triglyceride, cholesterol, very low density lipoprotein (VLDL), glucose and insulin. We found that the vitamin significantly reduced (P=0.005) plasma triacylglycerol and VLDL concentrations. Biotin produced the following changes (mean of absolute differences between 0 and 28 day treatment+/−S.E.M.): a) triacylglycerol −0.55+/−0.2 in the diabetic group and −0.92+/−0.36 in the nondiabetic group; b) VLDL: −0.11+/−0.04 in the diabetic group and −0.18+/−0.07 in the nondiabetic group. Biotin treatment had no significant effects on cholesterol, glucose and insulin in either the diabetic or nondiabetic subjects. We conclude that pharmacological doses of biotin decrease hypertriglyceridemia. The triglyceride-lowering effect of biotin suggests that biotin could be used in the treatment of hypertriglyceridemia.

A statistically significant inverse association was generally found between plasma total lipid, cholesterol, or phospholipid and biotin status of 300-day-old male inbred BHE (IN-BHE) rats. Plasma, liver, and carcass lipid of both sexes generally had a significant direct association with liver lactate dehydrogenase activity; an inverse association in males resulted with improved biotin status. Elevated plasma lactate indicative of anaerobic glycolysis was found. It is proposed that an increased reductive environment—a consequence of accumulated NADH—could account for enhanced triglyceride synthesis and that this effect could explain the obesity in the IN-BHE rats. After the injection of 300 mug of biotin, plasma levels of lactate and pyruvate fell in male rats, indicating a stimulatory effect of biotin upon the oxidative pathways in these animals.

Dyslipidemia, often found in type 2 diabetes mellitus (T2DM) patients, plays an important role in the progression of cardiometabolic syndrome. Two essential nutrients, chromium and biotin, may maintain optimal glycemic control. A randomized, double-blind placebo-controlled trial (N=348; chromium picolinate and biotin combination [CPB]: 226, placebo: 122; T2DM participants with hemoglobin A1c [HbA1c]>or=7%) evaluated the effects of CPB on lipid and lipoprotein levels. Participants were randomly assigned (2:1 ratio) to receive either CPB (600 microg chromium as chromium picolinate and 2 mg biotin) or a matching placebo once daily for 90 days. Statistical analyses were conducted in all eligible participants. Subsequent supplemental analyses were performed in T2DM participants with hypercholesterolemia (HC) and in those using stable doses of statins. In the primary analysis, CPB lowered HbA1c (P<0.05) and glucose (P<0.02) significantly compared with the placebo group. No significant changes were observed in other lipid levels. In participants with HC and T2DM, significant changes in total cholesterol and low-density lipoprotein cholesterol (LDL-C) levels and atherogenic index were observed in the CPB group (P<0.05). Significant decreases in LDL-C, total cholesterol, HbA1c, and very low-density cholesterol levels (P<0.05) were observed in the CPB group taking statins. CPB treatment was well tolerated with no adverse effects, dissimilar from those associated with placebo. These data suggest that intervention with CPB improves cardiometabolic risk factors.

Chromium functions as a cofactor for insulin. It binds to the insulin receptor and potentiates many, and perhaps all, of its functions (Boyle et al., supra.). These functions include, but are not limited to, the regulation of carbohydrate and lipid metabolism. The introduction of inorganic chromium compounds per se into individuals is not particularly beneficial. Chromium must be converted endogenously into an organic complex or must be consumed as a biologically active molecule. Only about 0.5% of ingested inorganic chromium is assimilated into the body (Recommended Daily Allowances, Ninth Revised Edition, The National Academy of Sciences, page 160, 1980). Only 1-2% of most organic compounds is assimilated into the body. U.S. Pat. No. Re. 33,988 discloses that when selected essential metals, including chromium, are administered to mammals as exogenously synthesized coordination complexes of picolinic acid, they are directly available for absorption without competition from other metals. These complexes are safe, inexpensive, biocompatible and easy to produce. These exogenously synthesized essential metal coordination complexes of picolinic acid (pyridine-2-carboxylic acid) have the following structural formula:

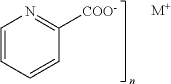

Curcumin (1) is a natural product isolated in abundance from Curcuma species, in particular, Curcuma longa (Zingiberaceae) Linn. See Park and Kim, /. Nat. Prod. 65: 1227 (2002) and U.S. Pat. No. 5,861,415. Recently, it has attracted considerable attention due to its antioxidant (Kawanishi et al., Antioxid. Redox Signal. 7: 1728 (2005)), anti-inflammatory (Chianani-Wu, /. Altern. Complement Med. 9: 161 (2003)), antiviral (Vajragupta et al., Bioorg. Med. Chem. Lett. 15: 3364 (2005) and Ranjan et al., /. Sur. Res. 87: 1 (1999)), antifungal (Kim et al., /. Agric. Food Chem. 51: 1578 (2003)), antibacterial (Foryst-Ludwig et al., Biochem. Biophys. Res. Commun. 316: 1065 (2004)), anticancer (Lee et al., Antioxid. Redox Signal. 7: 1612 (2005)), chemo preventive activities (Duvoix et al., Cancer Lett. 223: 181 (2005)), and in particular, its ability to protect neuronal cells from beta-amyloid insult (Park and Kim, /. Nat. Prod. 65: 1227 (2002) and Yang et al., /. Biol. Chem. 280, 5892 (2005)), suggesting a potentially important drug candidate to prevent and/or treat Alzheimer's Disease.

Bioperin, also known as piperine, is a black pepper extract that is a known inhibitor of glucuronidase in the gas-trointestinal tract and in the liver. Bioperin is also a stimulator of thennogenesis. Bioperin has been shown to increase the bioavailability of a large number of drugs, including propranolol. The properties of bioperin are described in U.S. Pat. No. 5,744,161 to Majeed et al. and U.S. Pat. No. 5,972,382 to Majeed et al. both of which are incorporated herein in their entirety by this reference.

As indicated above, one aspect of the invention is a composition including both pantethine and berberine. This invention provides the combination of pantethine and berberine as a new pharmaceutical remedy with beneficial therapeutic activities to the lipid disorders associated with cardiovascular disease and diabetes. The specific benefits of this combination include, among other things: a more rapid onset of action as compared to each of the individual ingredients and a synergistic improvement in the lipid profile of patients.

Thus, embodiments of the present invention are directed to compounds that have an enhanced therapeutic effect and in a preferred embodiment, a synergistic effect. These compounds are administered in combination, which is understood to mean that the compounds are present in a patient at least for some common period of time. The effect of the individual compound may be therapeutic or otherwise. However, when the compound is combined with at least one other compound (in this case pantethine with berberine), i.e. its synergistic partner or pair, the resulting effect is increased relative to the effect of the compounds individually. According to one aspect of the present invention, the combination of compounds, or a pharmaceutically acceptable form thereof, is administered to a patient, including a human, such that the effect of the compounds is increased compared to the effect of the compounds if administered individually. That is, the compounds produce a synergistic effect when administered or applied as a combination, thereby producing a more efficacious therapy or result. One example of the combinations of the present invention includes combinations of drugs or other therapeutic or agricultural agents that exhibit a synergistic effect when administered in combination. Embodiments of the present invention are also directed to methods of using the combination of compounds depending on the synergistic effect produced.

According to further embodiments of the invention, agents or compounds exhibiting an enhanced therapeutic effect, and preferably a synergistic effect, when administered are provided in a kit. In one aspect, the kit includes the compounds forming the combination, a pharmaceutically acceptable carrier, and optionally, instructions for use. The compounds may be provided in separate formulations for administration separately for interaction within an organism or the compounds may be combined into a single formulation for administration. Alternatively, the kit may provide the compounds in a single formulation for administration. It is important to understand that the enhanced and synergistic effects may not be dependent upon the manner in which the compounds are administered, so long as the compounds are able to act in the organism after administration to produce an enhanced and/or synergistic effect.

The invention also covers a range of berberine concentrations from about 50 mg to about 1000 mg in combination with pantethine range of concentrations from about 50 mg to about 2000 mg suitable for consumption on a daily basis. More preferably, the range of concentration of berberine is from about 100 mg to about 700 mg in combination with from about 100 mg to about 1200 mg of pantethine. Even more preferably, the range of concentration of berberine is from about 250 mg to about 500 mg in combination with from about 250 mg to about 750 mg of pantethine. The combination can be used via oral, sublingual, intranasal, transcutaneous, parenteral, or intraarticular routes of administration.

Accordingly, in general, one embodiment of the present invention is a pharmaceutical composition comprising: (1) a therapeutically effective quantity of berberine; and (2) a therapeutically effective quantity of pantethine, in a unit dose. Typically, the pharmaceutical composition further comprises a pharmaceutically acceptable carrier, diluents or excipient. In general, the pharmaceutical composition typically comprises from about 250 mg to about 400 mg, of berberine and from about 250 mg to about 600 mg of pantethine as a unit dose.

The pharmaceutical composition can be formulated for oral, sublingual, intranasal, transcutaneous, parenteral, or intraarticular routes of administration.

Typically, the pharmaceutical composition is formulated for the treatment of a disease or condition selected from the group consisting of hypercholesterolemia, hyperlipidemia, elevated triglycerides, syndrome X (metabolic syndrome), hyperglycemia, diabetes, cardiovascular disease and the like as well as disorders of lipid metabolism with failed response to standard therapies (for example statins) or any other disease or condition associated with lipids and lipid metabolism.

Pharmaceutically acceptable carriers, diluents, or recipients are agents which are not biologically or otherwise undesirable, i.e., the agents can be administered to a subject along with the berberine and pantethine, or other combinations of active ingredients as described herein without causing any undesirable biological effects or interacting in a deleterious manner with any of the components of the pharmaceutical composition in which it is contained. Pharmaceutically acceptable carriers enhance or stabilize the composition, or can facilitate preparation of the composition. Pharmaceutically acceptable carriers include solvents, dispersion media, coatings, antibacterial and antifungal agents, isotonic and absorption delaying agents, and the like that are physiologically compatible. The pharmaceutically/pharmaceutically acceptable carrier should be suitable for various routes of administration described herein.

Pharmaceutical compositions of the invention can be prepared in accordance with methods well known and routinely practiced in the art. See, e.g., Remington: The Science and Practice of Pharmacy, Mack Publishing Co., 20th ed., 2000; m1dSustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymers, lactide/glycolide copolymers, or polyoxyethylene-polyoxypropylene copolymers may be used to control the release of the compounds. Other potentially useful parenteral delivery systems for molecules of the invention include ethylene-vinyl acetate copolymer particles, osmotic pumps, implantable infusion systems, and liposomes.

Preparations formulated for oral administration may be in the form of tablets, drops, capsules, transbuccal patches or solutions. The pharmaceutical compositions contemplated by the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active ingredients in water-soluble form. Additionally, suspensions of the active ingredients can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or modulators which increase the solubility of the active ingredients to allow for the preparation of highly concentrated solutions. Pharmaceutical preparations for oral use can be obtained by combining the active modulators with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating modulators may be added, such as the cross-linked polyvinyl pyrrolidone, agar, or alginic acid or a salt thereof such as sodium alginate.

Dragee cores are provided with suitable coatings. For tins purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different doses of active ingredients.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added.

Other ingredients such as stabilizers, for example, antioxidants such as sodium citrate, ascorbyl palmitate, propylgallate, reducing agents, ascorbic acid, vitamin E, sodium bisulfate, butylated hydroxytoluene, BHA, acetylcysteine, monothioglycerol, phenylanaphthylamine, or lecithin can be used. Also, chelators such as EDTA can be used. Other ingredients that are conventional in the area of pharmaceutical compositions and formulations, such as lubricants in tablets or pills, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, particularly as described above. For administration of any of the compounds used in the present invention, preparations should meet sterility, pyrogenicity, general safety, and purity standards as required by the FDA Office of Biologics Standards or by other regulatory organizations regulating drugs.

Sustained-release formulations or controlled-release formulations are well-known in the art. For example, the sustained-release or controlled-release formulation can be (1) an oral matrix sustained-release or controlled-release formulation; (2) an oral multilayered sustained-release or con-trolled-release tablet formulation; (3) an oral multiparticulate sustained-release or controlled-release formulation; (4) an oral osmotic sustained-release or controlled-release formulation; (5) an oral chewable sustained-release or controlled-release formulation; or (6) a dermal sustained-release or controlled-release patch formulation.

The pharmacokinetic principles of controlled drug delivery are described, for example. in B. M. Silber et al., “Phannacokinetic/Phannacodynamic Basis of Controlled Drug Delivery” in Controlled Drug Delivery: Fundamentals and Applications (J. R. Robinson & V. H. L. Lee, eds, 2d ed., Marcel Dekker, New York, 1987), ch. 5, pp. 213-251, incorporated herein by tins reference.

One of ordinary skill in the art can readily prepare formulations for controlled release or sustained release comprising the active ingredients by modifying the formulations described above, such as according to principles disclosed in V. N. K. Li et al., “Influence of Drug Properties and Routes of Drug Administration on the Design of Sustained and Con-trolled Release Systems” in Controlled Drug Delivery: Fundamentals and Applications (J. R. Robinson & V. H. L. Lee, eds, 2d ed., Marcel Dekker, New York, 1987), ch. 1, pp. 3-94, incorporated herein by this reference. This process of preparation typically takes into account physicochemical properties of the active ingredients such as aqueous solubility, partition coefficient, molecular size, stability of the active ingredients, and binding of the active ingredients to proteins and other biological macromolecules. Tins process of preparation also takes into account biological factors, such as absorption, distribution, metabolism, duration of action, the possible existence of side effects, and margin of safety, for the active ingredients. Accordingly, one of ordinary skill in the art could modify the formulations in order to incorporate the active ingredients into a formulation having the desirable properties described above for a particular application.

Another embodiment of the present invention, as described above, includes bioperin in a therapeutically effective quantity in the unit dose. The bioperin is included in a therapeutically effective quantity to improve bioavailability of the berberine and pantethine. A therapeutically effective quantity of bioperin is typically from about 0.1 mg to about 20 mg per unit dose, more preferably from about 1 mg to about 10 mg per unit dosage.

Therefore, in general, this embodiment of the present invention is a pharmaceutical composition comprising: (1) a therapeutically effective quantity of berberine; (2) a therapeutically effective quantity of pantethine; and (3) a therapeutically effective quantity of bioperin, in a unit dose.

In a preferred embodiment the pharmaceutical composition comprises from about 250 mg to about 400 mg of berberine, and from about 250 mg to about 600 mg of pantethine as a unit dose combined from about one or more of from about 1 mcg to about 500 mcg of chromium polynicotinate, from about 0.1 mg to about 50 mg of biotin, from about 50 mg to about 2000 mg of curcumin, and from about 0.1 mg to about 20 mg of bioperin per unit dose. In a particularly preferred embodiment, the unit dosage form of the pharmaceutical composition comprises 250 mg of berberine, 300 mg of pantethine, 50 mcg of chromium, 2500 mcg of biotin, 250 mg of curcumin and 2.5 mg of bioperine.

Typically, as described above, this embodiment of the pharmaceutical composition also further comprises a pharmaceutically acceptable carrier, diluent, or excipient.

Yet another embodiment of the present invention is the combination of berberine and pantethine described above, and also including one or more additional compounds selected from the group consisting of chromium polynicotinate or chromium picolinate, biotin, curcumin and/or bioperin in a therapeutically effective quantity in the unit dose. In general, this embodiment comprises (1) a therapeutically effective quantity of berberine, (2) a therapeutically effective quantity of pantethine; and (3) a therapeutically effective quantity of one or more of chromium polynicotinate, chromium picolinate, biotin, curcumin or bioperin in a unit dose.

Typically, the therapeutically effective amount comprises about 250 mg to about 400 mg of berberine, and from about 250 mg to about 600 mg of pantethine as a unit dose combined from about one or more of from about 1 mcg to about 500 mcg of chromium picolinate, from about 0.1 mg to about 50 mg of biotin, from about 50 mg to about 2000 mg of curcumin, and from about 0.1 mg to about 20 mg of bioperin per unit dose. In a particularly preferred embodiment, the unit dosage form of the pharmaceutical composition comprises 250 mg of berberine, 300 mg of pantethine, 50 mcg of chromium, 2500 mcg of biotin, 250 mg of curcumin and 2.5 mg of bioperine.

Yet another aspect of the present invention is a method for treating a patient who has a condition selected from the group consisting of hypercholesterolemia, hyperlipidemia, elevated triglycerides, syndrome X (metabolic syndrome), hyperglycemia, diabetes, cardiovascular disease and the like as well as disorders of lipid metabolism with failed response to standard therapies (for example statins) or any other disease or condition associated with lipids and lipid metabolism by administering a therapeutically effective amount of one or more of the pharmaceutical compositions of the present invention. In general, this method comprises the step of administering a therapeutically effective dose of a pharmaceutical composition according to the present invention, as described above, to the patient to treat the patient.

### EXAMPLES OF THE INVENTION

### Example 1

In order to determine the efficacy of the present invention, a first trial was conducted. Five patients with hyperlipidemia were enrolled in the pilot clinical trial. All patients were recruited from IFSMED patient's registry. Each patient was initially treated with pantethine 300 mg PO BID for 6 weeks and subsequently treated with a combination of pantethine 300 mg PO BID and berberine 250 mg PO BID. Total cholesterol, LDL, HDL and triglycerides were measured in patient's sera (fasting) using chemical analyzer Beckman Coulter AU 480 before enrollment into the study, after completion of the 6-week consumption of the pantethine alone and, finally, after completion of the 6-week consumption of the pantethine/berberine combination. The results of the study are show in Table 1, below.

TABLE 1
Effects of Pantethine in comparison to Panthetine in combination with
Berberine and further in comparison to baseline values of total
cholesterol, LDL, triglycerides and HDL in five patients.

| | | | Pantethine + |
|---|---|---|---|
| | Baseline | Pantethine | Berberine |
| PATIENT NO. 1: | | | |
| a 67 Year Old | | | |
| Female | | | |
| total cholesterol | 187 | 169 | 142 |
| mg/dl | | | |
| (N 100-199) | | | |
| LDL mg/dl | 82 | 75 | 66 |
| (N 0-99) | | | |
| triglycerides | 312 | 228 | 168 |
| mg/dl (N 0-149) | | | |
| HDL mg/dl (N | 43 | 48 | 42 |
| >39) | | | |
| PATIENT NO. 2: | | | |
| a 70 Year Old | | | |
| Male | | | |
| total cholesterol | 311 | 279 | 201 |
| mg/dl | | | |
| (N 100-199) | | | |
| LDL mg/dl | 191 | 177 | 113 |
| (N 0-99) | | | |
| triglycerides | 282 | 237 | 171 |
| mg/dl (N 0-149) | | | |
| HDL mg/dl (N | 39 | 38 | 41 |
| >39) | | | |
| PATIENT NO. 3: | | | |
| a 57 Year Old | | | |
| Female | | | |
| total cholesterol | 211 | 181 | 153 |
| mg/dl | | | |
| (N 100-199) | | | |
| LDL mg/dl | 143 | 121 | 92 |
| (N 0-99) | | | |
| triglycerides | 199 | 164 | 132 |
| mg/dl (N 0-149) | | | |
| HDL mg/dl (N | 41 | 44 | 48 |
| >39) | | | |
| PATIENT NO. 4: | | | |
| a 55 Year Old | | | |
| Female | | | |
| total cholesterol | 377 | 244 | 189 |
| mg/dl | | | |
| (N 100-199) | | | |
| LDL mg/dl | 219 | 203 | 128 |
| (N 0-99) | | | |
| triglycerides | 369 | 314 | 232 |
| mg/dl (N 0-149) | | | |
| HDL mg/dl (N | 31 | 42 | 42 |
| >39) | | | |
| PATIENT NO. 5; | | | |
| an 81 Year Old | | | |
| Female | | | |
| total cholesterol | 198 | 162 | 134 |
| mg/dl | | | |
| (N 100-199) | | | |
| LDL mg/dl | 93 | 71 | 64 |
| (N 0-99) | | | |
| triglycerides | 274 | 220 | 176 |
| mg/dl (N 0-149) | | | |
| HDL mg/dl (N | 42 | 43 | 46 |
| >39) | | | |

As can be seen in Table 1, all five patients showed improvement in total serum cholesterol, LDL, triglycerides and HDL for Pantethine alone over baseline, and a more marked, and synergistically enhanced improvement of Pantethine in combination with Berberine versus Pantethine alone.

### Example 2

A second trial of the present invention was focused on the effect of a composition (“Composition 2”) according to the present invention (comprised of 250 mg of berberine, 300 mg of pantethine, 50 mcg of chromium, 2500 mcg of biotin, 250 mg of curcumin and 2.5 mg of bioperine in a unit dosage form) on total cholesterol, triglycerides, LDL and HDL in patients with hyperlipidemia. The trial was designed as an open-label clinical trial comparing the above mentioned analytes at the baseline and after 6 weeks of taking Composition 2. Eighteen participants (eight males with age range from 45 to 67 and ten females with the age range from 45-67) completed the trial. Table 1 shows percentage of participants who beneficially responded to Composition 2. Table 2 shows the actual changes in the analytes levels at the baseline and 6 weeks after administration of Composition 2. Results of the pilot study showed that Composition 2 according to the present invention significantly lowers the level of total cholesterol and LDL and demonstrates a trend toward lowering of triglycerides.

TABLE 1
Shows percentage of participants who
beneficially responded to Composition 2.

| RESPONSES | ABSOLUTE NUMBER | % |
|---|---|---|
| CHOLESTEROL | 13/18 | 72.2 |
| TRIG | 14/18 | 77.8 |
| LDL | 15/18 | 83.3 |
| HDL | 9/18 | 50.0 |

TABLE 2
Shows the actual changes in the analytes levels at the baseline and 6
weeks after administration of Composition 2

| | | | T-TEST | T-TEST |
|---|---|---|---|---|
| ANALYTE | BASELINE | WEEK 6 | PAIRED | HOMOSCEDASTIC |
| CHOLESTEROL | 243.5 | 218.7 | 0.00075* | 0.028* |
| TRIGLYCERIDES | 137 | 124.4 | 0.071 | 0.303 |
| LDL | 155 | 139.4 | 0.0063* | 0.0862 |
| HDL | 59.9 | 58.2 | 0.176 | 0.376 |

*STATISTICALLY SIGNIFICANT DIFFERENCES

As used herein, the terms “treating” or similar terminology do not imply a cure for any of the diseases or conditions described above, i.e., hypercholesterolemia, hyperlipidemia, elevated triglycerides, syndrome X (metabolic syndrome), hyperglycemia, diabetes, cardiovascular disease and the like as well as disorders of lipid metabolism with failed response to standard therapies (for example statins) or any other disease or condition associated with lipids and lipid metabolism; rather, this terminology is used to refer to any clinically detectable improvement in the disease or condition being treated or alleviated. As used herein, the term “therapeutically effective amount” refers to the amount of a therapy (e.g., a prophylactic or therapeutic agent) which is sufficient to treat any of the diseases or conditions described above.

The pharmaceutical composition in the methods of the invention is typically administered to a subject in an amount that is sufficient to achieve the desired therapeutic effect in a subject in need thereof. The selected dosage level for pharmaceutical compositions according to the present invention depends upon a variety of pharmacokinetic factors including the concentration of the active agents in the pharmaceutical composition, the route of administration, the frequency of administration, the rate of excretion of the active agents, the severity of the condition, other health considerations affecting the subject, and the status of liver and kidney function of the subject. It also depends on the duration of the treatment, other digs; compounds and/or materials used in combination with pharmaceutical compositions according to the present invention, as well as the age, weight, condition, general health and prior medical history of the subject being treated, and like factors. Methods for determining optimal dosages are described in the art, e.g., Remington: The Science and Practice of Pharmacy, Mack Publishing Co., 20th ed., 2000.

Pharmaceutical compositions and methods according to the present invention provide improved treatment of modalities for a number of diseases and conditions, such as hypercholesterolemia, hyperlipidemia, elevated triglycerides, syndrome X (metabolic syndrome), hyperglycemia, diabetes, cardiovascular disease and the like as well as disorders of lipid metabolism with failed response to standard therapies (for example statins) or any other disease or condition associated with lipids and lipid metabolism. They are well tolerated, have few side effects, and can he used with other medications for these diseases and conditions as well as lifestyle changes such as improved diet, exercise programs, and programs of relaxation and stress reduction.

Treatment methods according to the present invention possess industrial applicability for the preparation of a medicament for the treatment of diseases and conditions such as hypercholesterolemia, hyperlipidemia, elevated triglycerides, syndrome X (metabolic syndrome), hyperglycemia, diabetes, cardiovascular disease and the like as well as disorders of lipid metabolism with failed response to standard therapies (for example statins) or any other disease or condition associated with lipids and lipid metabolism.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore. be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

Throughout this application, various publications, patents, and/or patent applications are referenced in order to more fully describe the state of the art to which this invention pertains. The disclosures of these publications, patents, and/or patent applications are herein incorporated by reference in their entireties, and for the subject matter for which they are specifically referenced in the same or a prior sentence, to the same extent as if each independent publication, patent, and/or patent application was specifically and individually indicated to be incorporated by reference.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A pharmaceutical composition comprising:
(a) a therapeutically effective quantity of berberine; and
(b) a therapeutically effective quantity of pantethine, in a unit dose.

2. The pharmaceutical composition of claim 1 further comprising a pharmaceutically acceptable carrier, diluent, or excipient.

3. The pharmaceutical composition of claim 1 wherein the composition comprises from about 50 mg to about 1000 mg of berberine as a unit dose.

4. The pharmaceutical composition of claim 1 wherein the composition comprises from about 50 mg to about 2000 mg of pantethine as a unit dose.

5. The pharmaceutical composition of claim 1 further comprising one or more of chromium polynicotinate or chromium picolinate, biotin, curcumin or bioperin.

6. The pharmaceutical composition of claim 1 wherein the pharmaceutical composition is formulated for the treatment of a disease or condition selected from the group consisting of hypercholesterolemia, hyperlipidemia, elevated triglycerides, syndrome X (metabolic syndrome), hyperglycemia, diabetes, cardiovascular disease, disorders of lipid metabolism and diseases associated with lipids.

7. The pharmaceutical composition of claim 6 wherein the composition is formulated for the treatment of a disease or condition where the disease or condition has failed to respond to one or more existing standard therapies.

8. A pharmaceutical composition comprising:
(a) a therapeutically effective quantity of berberine;
(b) a therapeutically effective quantity of pantethine; and
(c) a therapeutically effective quantity of one or more of curcumin, chromium polynicotinate, and bioperin, in a unit dose.

9. The pharmaceutical composition of claim 8 further comprising a pharmaceutically acceptable carrier, diluent, or excipient.

10. The pharmaceutical composition of claim 8 wherein the composition comprises from about 50 mg to about 1000 mg of berberine as a unit dose.

11. The pharmaceutical composition of claim 8 wherein the composition comprises from about 50 mg to about 2000 mg of pantethine as a unit dose.

12. The pharmaceutical composition of claim 8 wherein the composition comprises from about 1 mcg to about 500 mcg of chromium polynicotinate, from about 0.1 mg to about 50 mg of biotin, from about 50 mg to about 2000 mg of curcumin, and from about 0.1 mg to about 20 mg of bioperin per unit dose.

13. The pharmaceutical composition of claim 8 wherein the composition comprises from about 50 mg to about 1000 mg of berberine, from about 50 mg to about 2000 mg of pantethine, and from about 500 mcg of chromium polynicotinate, from about 0.1 mg to about 50 mg of biotin, from about 50 mg to about 2000 mg of curcumin, and from about 0.1 mg to about 20 mg of bioperin per unit dose.

14. The pharmaceutical composition of claim 8 wherein the pharmaceutical composition is formulated for the treatment of a disease or condition selected from the group consisting of hypercholesterolemia, hyperlipidemia, elevated triglycerides, syndrome X (metabolic syndrome), hyperglycemia, diabetes, cardiovascular disease, disorders of lipid metabolism and diseases associated with lipids.

15. A method of treating a patient who has a condition selected from the group consisting of hypercholesterolemia, hyperlipidemia, elevated triglycerides, syndrome X (metabolic syndrome), hyperglycemia, diabetes, cardiovascular disease, disorders of lipid metabolism and diseases associated with lipids. comprising the step of administering a therapeutically effective dose of the pharmaceutical composition of claim 1 to the patient to treat the patient.

16. The method of treating a patient of claim 15 wherein the effect of the treatment on the condition is synergistic.

17. A method of treating a patient who has a condition selected from the group consisting of hypercholesterolemia, hyperlipidemia, elevated triglycerides, syndrome X (metabolic syndrome), hyperglycemia, diabetes, cardiovascular disease, disorders of lipid metabolism and diseases associated with lipids. comprising the step of administering a therapeutically effective dose of the pharmaceutical composition of claim 8 to the patient to treat the patient.
